(19) United States
(12) Patent Application Publication
(10) Pub. No.: US 2012/0101379 A1
(43) Pub. Date: Apr. 26, 2012

(54) ULTRASOUND DIAGNOSTIC APPARATUS

(75) Inventor: Yoshiki KATOU, Tokyo (JP)

(73) Assignee: KONICA MINOLTA MEDICAL & GRAPHIC, INC., Tokyo (JP)

(21) Appl. No.: 13/274,465

(22) Filed: Oct. 17, 2011

(30) Foreign Application Priority Data
Oct. 21, 2010 (JP) JP2010-236029

(51) Int. Cl. A61B 8/00

(52) U.S. Cl. 600/437

(57) ABSTRACT

The ultrasound probe includes an error correcting code generating unit to generate error correcting codes corresponding to the ultrasound data, adds the error correcting codes generated by the error correcting code generating unit to the ultrasound data so as to produce transmission data, and transmits the transmission data to the processing unit. The processing unit includes an error correcting unit to perform error correction processing for the ultrasound data based on the error correcting codes contained in the transmission data, and an error ratio detecting unit to detect an error ratio of data in the ultrasound data contained in the transmission data, and transmits information corresponding to the error ratio detected by the error ratio detecting unit to the ultrasound probe. The error correcting code generating unit changes a ratio of error correcting codes to the ultrasound data in accordance with the error ratio received by the ultrasound probe.

### CROSS REFERENCE TO RELATED APPLICATION

This application is based on Japanese Patent Application No. 2010-236029 filed on Oct. 21, 2010, in Japanese Patent Office, the entire content of which is hereby incorporated by reference.

### BACKGROUND OF THE INVENTION

The present invention relates to an ultrasound diagnostic apparatus. Conventionally, in the known wireless type ultrasound diagnostic apparatuses, ultrasound data acquired by ultrasound probes are transmitted wirelessly to the apparatus main bodies.

In such the ultrasound diagnostic apparatuses, error correcting codes are added to the data wirelessly transmitted by the ultrasound probes, so that even when the data are damaged on the transmission paths, the data are restored by the apparatus main bodies (for example, refer to Patent Document 1). Further, in the ultrasound diagnostic apparatuses, attention is focused on the point that a data restoring ability can be improved more as the error correcting codes are added more, and the ratio of the error correcting codes to the transmission data is changed in accordance with the degree of the importance of data to be transmitted.

Patent document 1: Japanese Unexamined Patent Publication No. 2009-291515 official report Incidentally, in order to display ultrasound diagnostic images in real time in the wireless type ultrasound diagnostic apparatus, reception signals of the ultrasound wave acquired by the ultrasound probe are required to be converted into ultrasound data by an A/D conversion and to be transmitted wirelessly in real time to the processing unit. In the case where the sampling frequency of ultrasound data is 60 MHz, the number of transducers disposed in the ultrasound probe is 128, and the number of bits per one oscillator is 12, if the data acquired in the above case are transmitted wirelessly in real time, the data transmission rate is required to be 92160 Mbps or more. With regard to this data transmission, in the ultrasound diagnostic apparatus described in the abovementioned Patent document 1, error correcting codes with the large number of bits are always added to the important data even in the good transmission state in which data are hardly damaged, which results in that the data transmission rate is lowered. Accordingly, there is fear to cause problems that the data transmission efficiency is not good, and further the data transmission rate always runs short. In this way, when the data transmission rate is lower than a required transmission rate, the shortage of the data transmission rate may be solved in such a way that, for example, ultrasound data are not transmitted for each scan with ultrasound waves and thinned out, and the thinned-out ultrasound data are transmitted. However, this thinned-out ultrasound data cause the lowering of a flame rate. Accordingly, in the case where the ultrasound diagnostic apparatus described in the abovementioned Patent document 1 is employed, although ultrasound diagnostic images are important, the images become always images with a low flame rate even in the good transmission state, and there is fear that proper diagnosis cannot be performed.

### SUMMARY OF THE INVENTION

An object of the present invention is to provide an ultrasound diagnostic apparatus which can transmit data with a proper data transmission rate in response to a transmission state.

The above object can be attained by the following ultrasound diagnostic apparatus on which one aspect of the present invention is reflected.

An ultrasound diagnostic apparatus comprising:
an ultrasound probe which outputs transmission ultrasound waves based on drive signals toward an object to be examined, receives ultrasound waves reflected from the object so as to acquire reception signals, and generates ultrasound data based on the acquired reception signals; and
a processing unit which produces an ultrasound diagnostic image based on the ultrasound data
wherein the ultrasound probe and the processing unit respectively includes a transceiver unit to transmit and receive data wirelessly,
wherein the ultrasound probe includes an error correcting code generating unit which generates error correcting codes corresponding to the ultrasound data, and the ultrasound probe adds the error correcting codes generated by the error correcting code generating unit to the ultrasound data so as to produce transmission data, and transmits the transmission data to the processing unit,
wherein the processing unit includes an error correcting unit which performs error correction processing for the ultrasound data contained in the received transmission data based on the error correcting codes contained in the transmission data, and an error ratio detecting unit which detects an error ratio of data in the ultrasound data contained in the transmission data, and the processing unit transmits information corresponding to the error ratio detected by the error ratio detecting unit to the ultrasound probe, and
wherein the error correcting code generating unit changes a ratio of error correcting codes to the ultrasound data in accordance with the information corresponding to the error ratio received by the ultrasound probe and generates error correcting codes.

### BRIEF DESCRIPTION OF THE DRAWINGS

FIG. 1 is a diagram showing the appearance structure of an ultrasound diagnostic apparatus in the first embodiment.

FIG. 2 is a block diagram showing the outline structure of an ultrasound probe in the first embodiment.

FIG. 3 is a diagram for explaining envelope data.

FIG. 4 is a block diagram showing the outline structure of an ultrasound diagnostic apparatus processing unit in the first embodiment.

FIGS. 5a and 5b each is a diagram for explaining a data configuration of transmission data.

FIG. 6 is a block diagram showing the outline structure of the ultrasound probe in the second embodiment.

FIG. 7 is a block diagram showing the outline structure of the ultrasound diagnostic apparatus processing unit in the second embodiment.

FIG. 8 is a block diagram showing the outline structure of the ultrasound probe in the third embodiment.

FIG. 9 is a block diagram showing the outline structure of the ultrasound diagnostic apparatus processing unit in the third embodiment.

FIGS. 10a, 10b, and 10c each is a diagram for explaining other examples of a data configuration of transmission data.

### DETAILED DESCRIPTION OF THE PREFERRED EMBODIMENT

Hereafter, an ultrasound diagnostic apparatus according to the embodiment of the present invention will be explained with reference to a drawing. However, the scope of the invention is not limited to the examples shown in the drawings. In the following description, structural parts which have the same function and structure respectively, are provided with the same reference symbols, and the description for them will be omitted.

### First Embodiment

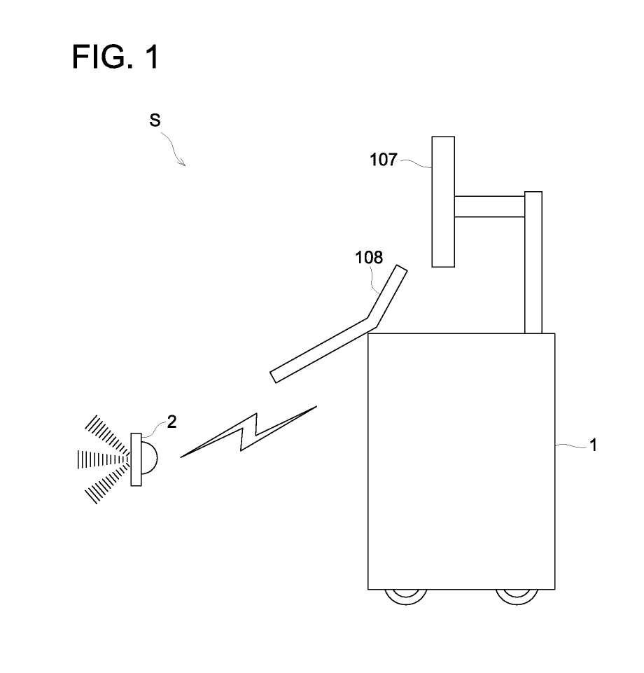

An ultrasound diagnostic apparatus S according to the first embodiment of the present invention includes a processing unit (an ultrasound diagnostic apparatus main body) 1 and a ultrasound probe 2 as shown in FIG. 1. The ultrasound probe 2 transmits ultrasound waves (transmitted ultrasound waves) to an examined object, such as a living body which is not illustrated, and receives the reflected waves (reflected ultrasound waves: echo) of the ultrasound wave reflected by this examined object. The ultrasound probe 2 is structured to be able to transmit date to and receive wirelessly data from the ultrasound diagnostic apparatus processing unit 1. As the wireless communication systems, any known system may be employed. However, in the present embodiment, for example, the system of the international standard “IEEE802.11n” is employed. The ultrasound probe 2 acquires reception signals being electrical signals from the received reflected ultrasound waves, converted the reception signals into data with a predetermined transmission format by an A/D conversion, and then, transmits wirelessly the data to the ultrasound diagnostic apparatus processing unit 1.

Based on the data transmitted from the ultrasound probe 2, the ultrasound diagnostic apparatus processing unit 1 makes an internal state in the examined object to an ultrasound diagnostic image, and displays it on the display unit 107. Moreover, the ultrasound diagnostic apparatus processing unit 1 includes an operation inputting unit 108, and can transmit information to the ultrasound probe 2 in accordance with operations of the operation inputting unit 108.

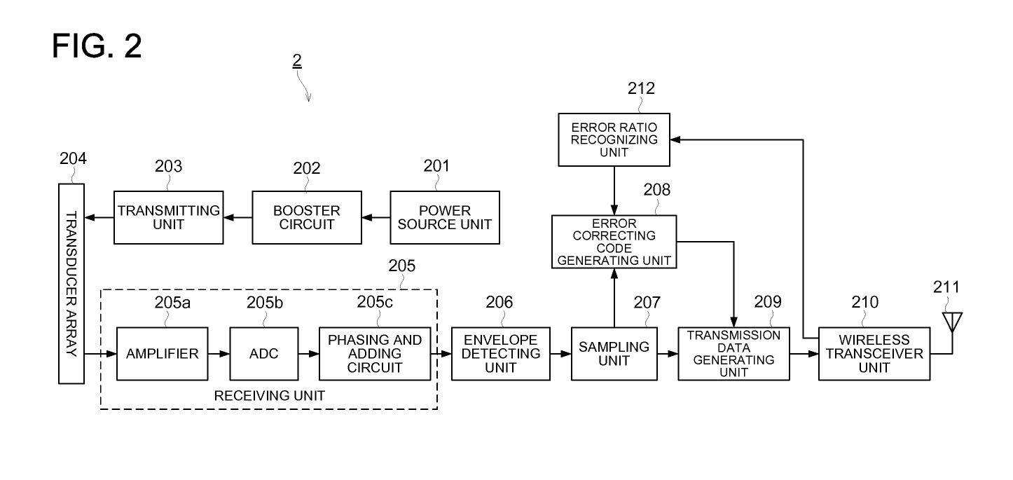

As shown in FIG. 2, the ultrasound probe 2 includes, for example, a power source unit 201, a booster circuit 202, a transmitting unit 203, an transducer array 204, a receiving unit 205, an envelope detecting unit 206, a sampling unit 207, an error correcting code generating unit 208, a transmission data generating unit 209, a wireless transceiver unit 210, an antenna 211, and a error ratio recognizing unit 212.

The power source unit 201 is constituted by, for example, batteries, and supplies a power source to respective units which constitutes the ultrasound probe 2. For example, when the ultrasound probe 2 is attached to a holder (not shown) of the ultrasound diagnostic apparatus processing unit 1, electric power is supplied to the power source unit 201. The booster circuit 202 is a circuit which is configured to raise a power source voltage supplied from the power source unit 201 to a voltage of 60 V to 150 V which can drive the ultrasound probe 2, and to supply the raised power voltage to the transmitting unit 203.

The transmitting unit 203 is a circuit configured to supply driving signals being electrical signals to the oscillator array 204, and to make the oscillator array 204 generate transmitted ultrasound waves.

In the oscillator array 204, a plurality of transducers each including a piezoelectric element is arranged in a one dimensional array form, and after outputting transmitted ultrasound waves, upon receipt of reflected ultrasound waves, the transmitting unit 203 outputs reception signals to the receiving unit 205. In this embodiment, the oscillator array 204 includes 192 transducers, for example. In this regard, the transducers may be arranged in a two-dimensional array form. Further, the number of transducers may be set up arbitrarily. Moreover, in this embodiment, although the linear electronic scan probe is adopted as the ultrasound probe 2, any type of an electronic scanning type or a mechanical scanning mariner may be adopted, and further any type of a linear scan type, a sector scanning type and a convex scan type may also be adopted. The transmitting unit 203 includes, for example, a transmitted BF (Beam Forming) control circuit, and sets a delay time in an individual path for a transmission timing of a driving signal for each vibrator, and focus a transmitted beam composed of transmitted ultrasound waves by delaying transmission of respective driving signals by the set delaying time.

The receiving unit 205 includes an amplifier 205a, an ADC (Analog/Digital Converter) 205b, and a phasing and adding circuit 205c, receives the reception signals output from the oscillator array 204, produces sound ray data, and outputs the sound ray data to the envelope detecting unit 206. The amplifier 205a is a circuit for amplifying the reception signals with respective predetermined amplification factors set beforehand for respective paths corresponding to the respective transducers. The ADC 205b is a circuit for carrying out the A/D conversion for the amplified reception signals. The phasing and adding circuit 205c is a circuit for giving the A/D-converted reception signals a delay time via respective paths corresponding to the respective transducers so as to adjust respective time phases, and for adding these (phasing addition) so as to produce sound ray data.

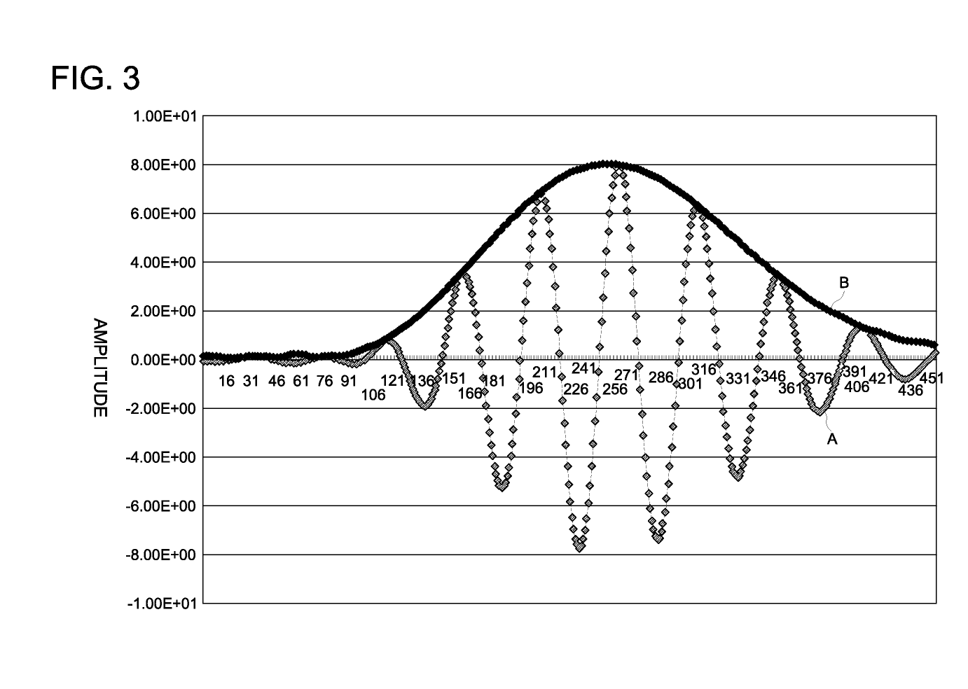

The envelope detecting unit 206 performs full wave rectification to the sound ray data output by the receiving unit 205, and obtains envelope data For example, when the sound ray data with a frequency of 4 MHz are sampled with a sampling frequency of 60 MHz, the sampled sound ray data are represented by a broken line A in FIG. 3. Then, the sampled sound ray data are subjected to the full wave rectification so as to extract an envelope curve, whereby envelope data indicated with a solid line B shown in FIG. 3 are obtained.

The sampling unit 207 conducts decimation for the envelope data obtained by the envelope detecting unit 206, whereby the envelope data are subjected to down sampling. In this embodiment, for example, the data rate of the envelope data is made to ⅛ by the down sampling. In this connection, the data rate after the down sampling may be set up arbitrarily. Further, the down sampling may not be performed. The sampling unit 207 divides the envelope data subjected to the down sampling into multi data with a predetermined data unit, and transmits the divided multi data to the error correcting code generating unit 208 and the transmission data generating unit 209.

The error correcting code generating unit 208 generates error correcting codes corresponding to the envelope data transmitted from the sampling unit 207 after the down sampling, and transmits the error correcting codes to the transmission data generating unit 209. As the error correcting codes, known codes, such as Hamming codes, BCH codes, and Reed Solomon codes, may be employable. In this embodiment, the error correcting code generating unit 208 changes the code length of error correcting codes in accordance with code length information from a later-mentioned error ratio recognizing unit 212 so as to change the ratio of error correcting codes to the envelope data for each of a transmission data unit (packet).

The transmission data generating unit 209 adds the error correcting codes transmitted from the error correcting code generating unit 208 to the data transmitted from the sampling unit 207, thereby producing transmission data, and transmits the transmission data to the wireless transceiver unit 210.

The wireless transceiver unit 210 applies a predetermined modulation processing to the transmission data transmitted from the transmission data generating unit 209, and transmits wirelessly the processed transmission data to the ultrasound diagnostic apparatus processing unit 1 via an antenna 211. Further, the wireless transceiver unit 210 receives via the antenna 211 later-mentioned error ratio information transmitted from the ultrasound diagnostic apparatus processing unit 1, demodulates the received error ratio information, and transmits the demodulated error ratio information to the error ratio recognizing unit 212. The wireless transceiver unit 210 changes the magnitude of a transmission output in accordance with the received error ratio information. That is, the wireless transceiver unit 210 is configured to improve the stability of transmission and reception of transmission data by changing the magnitude of a transmission output in proportion to the error ratio indicated by error ratio information. In this regard, the magnitude of a transmission output may be made constant.

The error ratio recognizing unit 212 analyzes the error ratio information transmitted from the wireless transceiver unit 210, and transmits code length information designating the code length of error correcting codes to the error correcting code generating unit 208. Upon receipt of this code length information, the error correcting code generating unit 208 generates error correcting code with a code length corresponding to the received code length information. When transmission data transmitted wirelessly from the ultrasound probe 2 is received by the ultrasound diagnostic apparatus processing unit 1, error ratio information is information indicating how many data are erroneous in ultrasound data (i.e., envelope data in this embodiment) contained in the transmission data That is, the error ratio information is information which shows the ratio of erroneous transmission data during the wireless transmission from the ultrasound probe 2 to the ultrasound diagnostic apparatus processing unit 1.

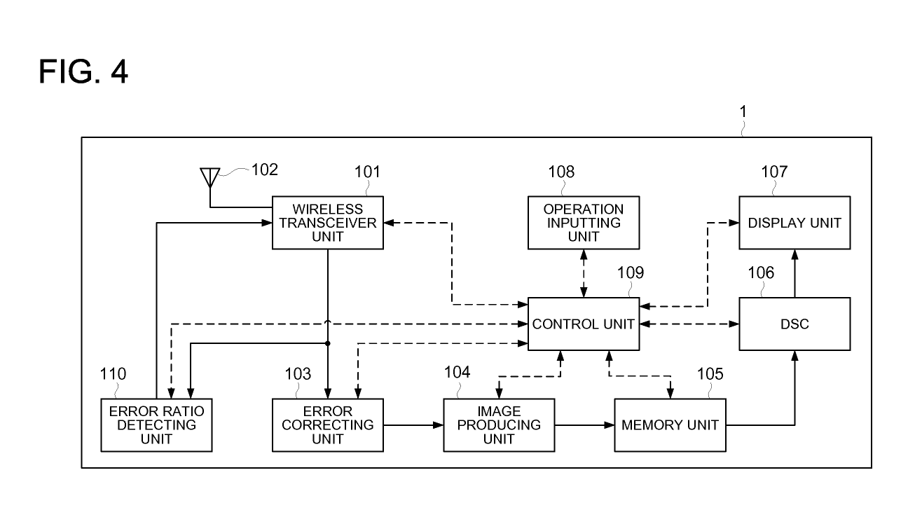

As shown in FIG. 4, the ultrasound diagnostic apparatus processing unit 1 includes, for example, a wireless transceiver unit 101, an antenna 102, an error correcting unit 103, an image producing unit 104, a memory unit 105, a DSC (Digital Scan Converter) 106, a display unit 107, an operation inputting unit 108, a control unit 109, and an error ratio detecting unit 110.

The wireless transceiver unit 101 receives transmission data transmitted wirelessly from the ultrasound probe 2 via the antenna 102, demodulates the transmission data, and transmits the demodulated transmission data to the error correcting unit 103 and the error ratio detecting unit 110. Further, the wireless transceiver unit 101 applies a predetermined modulation processing to the error ratio information transmitted from the error ratio detecting unit 110, and transmits wirelessly the processed error ratio information to the ultrasound probe 2 via the antenna 102.

The error correcting unit 103 performs error correction processing for the transmission data transmitted from the wireless transceiver unit 101. That is, the error correcting unit 103 performs error correction processing corresponding to the error correcting codes generated in the error correcting code generating unit 208 of the ultrasound probe 2. Even when errors are caused on a part of correction possible range contained in transmission data, this error correction processing makes it possible to restore the envelope data within a correction possible range by error correcting codes contained in the transmission data. The correction possible range of the correction possible range is determined by the code length of the error correcting codes. That is, the longer the code length is, the larger the correction possible range becomes. Successively, the error correcting unit 103 extracts envelope data from the transmission data subjected to the error correction processing, and transmits the extracted envelope data to the image producing unit 104. Further, as a result of the performed error correction processing, in the case where envelope data cannot be restored, the error correcting unit 103 transmits the envelope data to the image producing unit 104 as error processing without restoring this envelope data. With regard to the error processing, in place of the above embodiment, another embodiment may be employed. For example, the error correcting unit 103 notifies an error notice to the control unit 109, and the same image may be indicated on the display unit 107 until the error is canceled. Further, for example, a buffer is disposed in the error correcting unit 103 so as to interpolate the received envelope data until the error is canceled, and the error correcting unit 103 is configured to transmit the interpolated data to the image producing unit 104. Furthermore, in this embodiment, in the case where the envelope data cannot be restored, the error correcting unit 103 is configured to notify information to indicate this situation the control unit 109 in order to indicate warning on the display unit 107.

The image producing unit 104 generates B-mode image data based on the received envelope data. The B-mode image data expresses the strength of reception signals with brightness. Subsequently, the B-mode image data produced in the above ways are transmitted to the memory unit 105.

The memory unit 105 is constituted by, for example, semiconductor memories, such as DRAM (Dynamic Random Access Memory), and memorizes the B-mode image data transmitted from the image producing unit 104 by a frame unit. That is, the B-mode image data can be memorized as frame image data. Subsequently, the memorized frame image data is transmitted to the DSC 106 in accordance with control of the control unit 109.

The DSC 106 converts the frame image data received from the memory unit 105 into image signals corresponding to the scan mode by television signals, and outputs them to the display unit 107.

The display unit 107 is one of displays, such as a LCD (Liquid Crystal Display), a CRT (Cathode-Ray Tube) display, an organic electroluminescence (Electronic Luminescence) display, and a plasma display. In this connection, in replace of a display device, printing devices such as printers may be employed. The display unit 107 displays images on a display screen in accordance with the image signals output from the DSC 106. Further, upon receipt of information instructing to conduct warning from the control unit 109, the display unit 107 displays warning for telling the situation that ultrasound diagnostic images based on transmission data cannot be displayed. This display of warning is intended to display, for example, information for urging to confirm whether devices to influence wireless transmission of transmission data are disposed at surrounding areas, or information for telling merely that transmission and reception conditions for wireless transmission are not good. Accordingly, it is preferable to make the condition of wireless transmission to be recognizable for users. In this regard, in place of display of such information, for example, identification information, such as specific symbols may be displayed.

The operation inputting unit 108 is equipped with various types of switches, buttons, trackballs, mouse, and keyboards, for example, for performing input of commands to instruct start of diagnosis and data with regard to personal information of objects to be examined, and the operation inputting unit 108 outputs operation signals to the control unit 109.

The control unit 109 is constituted so as to include, for example, a CPU (Central Processing Unit), a ROM (Read Only Memory) and a RAM (Random Access Memory), and is configured to read out various processing programs, such as system programs memorized in the ROM, develops the read-out programs into the RAM, and conduct centralized control for operations of respective units of the ultrasound diagnostic apparatus S in accordance with the developed programs. The ROM is constituted by nonvolatile memories, such as semi-conductors, and is configured to memorize system programs corresponding to the ultrasound diagnostic apparatus S, various processing programs which can be executed on the system programs, and various data. These programs are stored with the form of program codes which can be read out by a computer, and the CPU executes operations in accordance with these program codes successively. The RAM forms work areas which memorize temporarily various programs executed by the CPU and data relating to these programs.

The error ratio detecting unit 110 performs error ratio detection processing for the transmission data transmitted from the wireless transceiver unit 101. That is, the error ratio detecting unit 110 detects an error ratio, which shows how many the errors of data are caused on the entire envelope data in envelope data, based on error correcting codes contained in transmission data Successively, the error ratio detecting unit 110 produces error ratio information which shows the detected error ratio, and outputs it to the wireless transceiver unit 101.

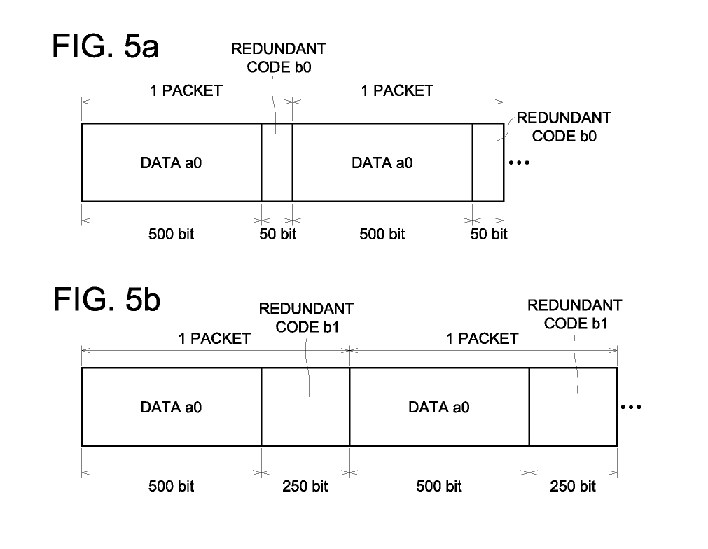

According to the ultrasound diagnostic apparatus S constituted as mentioned above, for example, under the environment where the ultrasound diagnostic apparatus S is not likely to receive interference of noises easily, since the possibility that transmission data are damaged so as to cause errors is low, an error ratio detected in the error ratio detecting unit 110 becomes very low. As a result, the error ratio information which shows that an error ratio is low is transmitted from the ultrasound diagnostic apparatus processing unit 1 to the ultrasound probe 2. Further, in the error correcting code generating unit 208, for example, as shown in FIG. 5 (a), error correcting codes (redundant codes) b0 with a data length of 50 bits are generated for the envelope data a0 composed of 500 bits, and these are synthesized by the transmission data generating unit 209 so as to form the data of one packet. Then, the envelope data a0 and the error correcting codes b0 are transmitted wirelessly to the ultrasound diagnostic apparatus processing unit 1.

Further, for example, on the condition that other devices are installed at surrounding areas so that the ultrasound diagnostic apparatus S tends to receive interference of noises, or under the environments that the distance between the ultrasound diagnostic apparatus processing unit 1 and the ultrasound probe 2 is far, the possibility that transmission data are damaged so as to cause errors becomes high, resulting in that an error ratio detected in the error ratio detecting unit 110 may become high. In such a case, the error ratio information showing that an error ratio is high is transmitted from the ultrasound diagnostic apparatus processing unit 1 to the ultrasound probe 2. Further, in the error correcting code generating unit 208, for example, as shown in FIG. 5 (b), redundant codes b1 with a data length of 250 bits are generated for the envelope data a0 composed of 500 bits, and these are synthesized by the transmission data generating unit 209 so as to form the data of one packet. Then, the envelope data a0 and redundant codes b1 are transmitted wirelessly to the ultrasound diagnostic apparatus processing unit 1.

In this way, according to this embodiment, in the case where the ultrasound diagnostic apparatus S is not likely to receive interference of noises easily and the error ratio in transmission data is low, since the wireless transmission is conducted in such a way that the code length of the error correcting codes added to envelope data is made small, the amount of data to he transmitted is decreased. Accordingly, a data transmission rate can be raised, and error correction can be performed appropriately. In this regard, in the case where the code length of transmission data is maintained, when an error ratio is low, it is possible to enlarge the data length of data to be transmitted at one time of data transmission. On the other hand, when an error ratio in transmission data becomes high, since the code length of the error correcting code added to envelope data is enlarged, error correction can be made to perform more certainly even under an environment where errors tend to be caused in envelope data. As a result, since data can be transmitted with a suitable data transmission rate in conformity with a transmission state, the transmission efficiency of data becomes good.

Further, according to this embodiment, since envelope data are made to be transmitted to the ultrasound diagnostic apparatus processing unit 1 from the ultrasound probe 2, the amount of data required for transmission can be made small, and a data transmission rate can be improved. That is, for example, in the case where sound ray data itself are transmitted wirelessly, in order to transmit the waveform data of reflected ultrasound waves as faithfully as possible, it may he preferable to make a sampling frequency to be 60 MHz or more. On the other hand, in the case where envelope data are transmitted, for example, as shown in FIG. 3, in the waveform formed by envelope data, a changed frequency is low as compared with the waveform formed by sound ray data. Accordingly, even if envelope data are subjected to down sampling, and then transmitted wirelessly, the faithfulness of the waveform can be maintained. Further, since a data transmission rate can be lowered, it is advantageous. For example, the total sum of data are obtained on the condition that the sampling frequency of sound ray data is 60 MHz, the number of transducers is 192, and the number of bits per one oscillator is 14, and then 22 bits data at high order in the total sum of data are subjected to ⅛ down sampling so as to obtain envelope data Subsequently, in the case where the resultant envelope data are transmitted wirelessly, the envelope data can be transmitted with a data transmission rate calculated as follows.

60×10⁶×22/8=0.165 (Gbps) (1)

Further, it may be preferable that at the time of sampling of the envelope data, a sampling unit 113 changes a sampling period in accordance with information corresponding to the error ration received by the ultrasound probe and generates ultrasound data. For example, when an error ration is low, since the code length of error correcting codes can be made small, the sampling period is made short (the sampling frequency is made high), so that the reproducibility of data can be made high. Meanwhile, when an error ration is high, since the code length of error correcting codes is required to be made large, the sampling period is made long (the sampling frequency is made low), so that the amount of data to be transmitted is reduced and the data transmission rate can be preferably improved.

### Second Embodiment

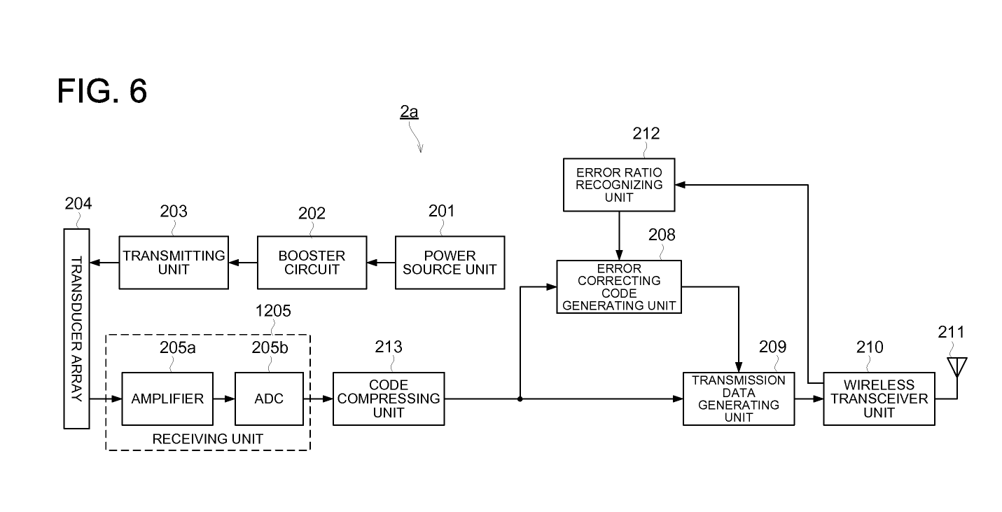

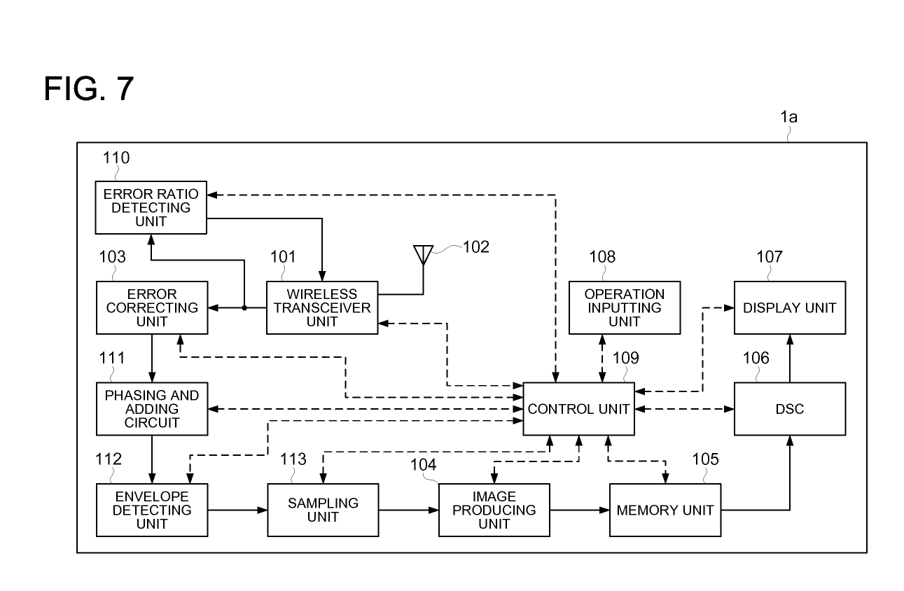

Referring to FIGS. 6 and 7, the second embodiment of the present invention will be described.

The ultrasound diagnostic apparatus S according to the second embodiment of the present invention is equipped with an ultrasound diagnostic apparatus processing unit 1a in place of the ultrasound diagnostic apparatus processing unit 1, and a ultrasound probe 2a in place of the ultrasound probe 2 in the first embodiment. The same structural parts as those in the first embodiment are provided with the same reference symbols, and the descriptions for them will be omitted.

Different from the first embodiment, the ultrasound probe 2a is configured to conduct amplification and A/D conversion for reception signals from the oscillator array 204 so as to produce data, compresses the produced data, adds error correcting codes to the compressed data, and then, transmits wirelessly them to the ultrasound diagnostic apparatus processing unit 1a.

More concretely, as shown in FIG. 6, the ultrasound probe 2a is constituted to be equipped with the power source unit 201, the booster circuit 202, the transmitting unit 203, the oscillator array 204, a receiving unit 1205, a code compressing unit 213, the error correcting code generating unit 208, the transmission data generating unit 209, the wireless transceiver unit 210, the antenna 211, and the error ratio recognizing unit 212.

Different from the first embodiment, the receiving unit 1205 is constituted such that the phasing and adding circuit is omitted. That is, the receiving unit 1205 amplifies reception signals by the amplifier 205a, conducts A/D conversion for the amplified reception signals by the ADC 205b, and outputs the resulting signals to the code compressing unit 213.

The code compressing unit 213 performs data compression for the reception signals, which were subjected to A/D conversion and output from the receiving unit 1205, by a known code compression technique. Successively, the code compressing unit 213 divides the data of the compressed reception signals into data groups with a predetermined data unit, and outputs them to the error correcting code generating unit 208 and the transmission data generating unit 209.

In the above-mentioned ways, the error correcting code generating unit 208 generates error correcting codes corresponding to the data of the compressed reception signals transmitted from the code compressing unit 213, and outputs them to the transmission data generating unit 209. As with the first embodiment, the error correcting code generating unit 208 generates error correcting codes with the code length corresponding to the code length information from the error ratio recognizing unit 212.

Hereafter, in the same ways as those in the first embodiment, transmission data are produced, and transmitted wirelessly to the ultrasound diagnostic apparatus processing unit 1a. Further, transmission of error ratio information transmitted from the ultrasound diagnostic apparatus processing unit 1a to the error ratio recognizing unit 212, and changing of the magnitude of the transmission output in conformity with the error ratio information are also performed.

Different from the first embodiment, the ultrasound diagnostic apparatus processing unit 1a conducts phasing addition and envelope detection for the data of the reception signals subjected to the error correction processing, further conducts down sampling, and thereafter, produces B-mode image data.

More concretely, as shown in FIG. 7, the ultrasound diagnostic apparatus processing unit 1a is constituted so as to be equipped with the wireless transceiver unit 101, the antenna 102, the error correcting unit 103, a phasing and adding circuit 111, an envelope detecting unit 112, a sampling unit 113, the image producing unit 104, the memory unit 105, the DSC 106, the display unit 107, the operation inputting unit 108, the control unit 109, and the error ratio detecting unit 110.

Since the phasing and adding circuit 111 and the envelope detecting unit 112 are the same with the phasing and adding circuit 205c and the envelope detecting unit 206 in the first embodiment, the detailed description for them will be omitted. As mentioned above, the sampling unit 113 conducts down sampling for the envelope data obtained by the envelope detecting unit 112, and thereafter, outputs the resulting data to the image producing unit 104.

Thus, eve in the second embodiment, error correcting codes with the code length according to an error ratio are added to data to be transmitted. Accordingly, data can be transmitted with a suitable data transmission rate in response to a transmission state.

Further, in the second embodiment, since the ultrasound diagnostic apparatus processing unit 1a is equipped with the phasing and adding circuit, the envelope detecting unit, and the sampling unit, a data transmission rate is inferior as compared with the ultrasound diagnostic apparatus S in the first embodiment. However, power consumption in the ultrasound probe 2a being a so-called wireless type can be suppressed, so that it becomes possible to endure even for continuous use for a long time.

In the second embodiment, it may be structured that the code compressing unit 213 is not disposed, and data of reception signals may be transmitted wirelessly without being compressed.

### Third Embodiment

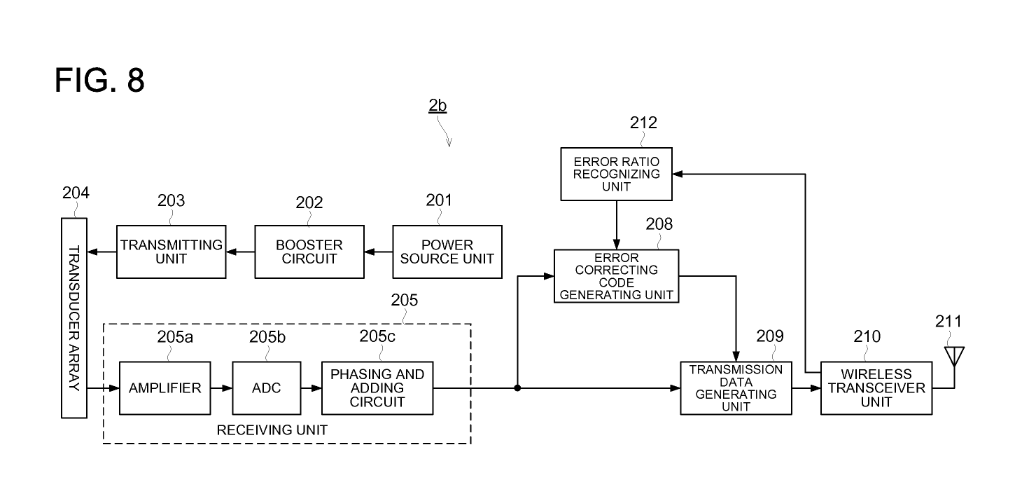

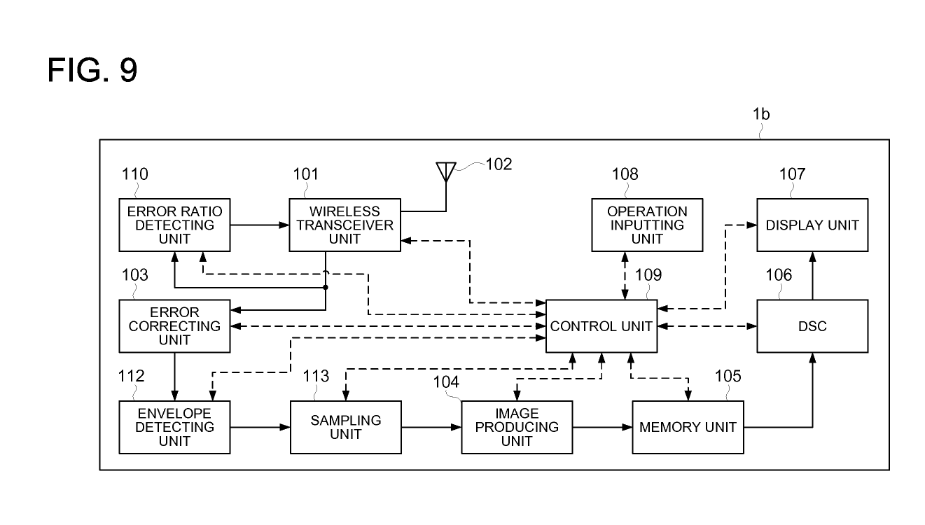

Referring to FIGS. 8 and 9, the third embodiment of the present invention will be described.

The ultrasound diagnostic apparatus S according to the third embodiment of the present invention is equipped with an ultrasound diagnostic apparatus processing unit 1b in place of the ultrasound diagnostic apparatus processing unit 1, and a ultrasound probe 2b in place of the ultrasound probe 2 in the first embodiment. The same structural parts as those in the first and second embodiments are provided with the same reference symbols, and the descriptions for them will be omitted.

Different from the second embodiment, the ultrasound probe 2b is configured to conduct amplification and A/D conversion for reception signals from the oscillator array 204 so as to produce data, further conducts phasing addition for the produced data, adds error correcting codes to the resulting data, and then, transmits wirelessly them to the ultrasound diagnostic apparatus processing unit 1b.

More concretely, as shown in FIG. 8, the ultrasound probe 2b is constituted to be equipped with the power source unit 201, the booster circuit 202, the transmitting unit 203, the oscillator array 204, the receiving unit 205, the error correcting code generating unit 208, the transmission data generating unit 209, the wireless transceiver unit 210, the antenna 211, and the error ratio recognizing unit 212.

As mentioned above in the first embodiment, the receiving unit 205 generates sound ray data, and thereafter, divides this sound ray data into data groups with a predetermined data unit, and transmits them to the error correcting code generating unit 208 and the transmission data generating unit 209.

As mentioned above, the error correcting code generating unit 208 generates error correcting codes corresponding to the sound ray data transmitted from the receiving unit 205, and transmits them to the transmission data generating unit 209. As with the first embodiment, the error correcting code generating unit 208 generates error correcting codes with the code length corresponding to the code length information from the error ratio recognizing unit 212.

Hereafter, in the same ways as those in the first embodiment, transmission data are produced, and transmitted wirelessly to the ultrasound diagnostic apparatus processing unit 1b. Further, transmission of error ratio information transmitted from the ultrasound diagnostic apparatus processing unit 1b to the error ratio recognizing unit 212, and changing of the magnitude of the transmission output in conformity with the error ratio information are also performed.

Different from the first embodiment, the ultrasound diagnostic apparatus processing unit 1b conducts envelope detection for the sound ray data subjected to the error correction processing, further conducts down sampling, and thereafter, produces B-mode image data.

More concretely, as shown in FIG. 9, the ultrasound diagnostic apparatus processing unit 1b is constituted so as to be equipped with the wireless transceiver unit 101, the antenna 102, the error correcting unit 103, the envelope detecting unit 112, the sampling unit 113, the image producing unit 104, the memory unit 105, the DSC 106, the display unit 107, the operation inputting unit 108, the control unit 109, and the error ratio detecting unit 110.

In this way, eve in the third embodiment, error correcting codes with the code length according to an error ratio are added to data to be transmitted. Accordingly, data can be transmitted with a suitable data transmission rate in response to a transmission state.

Further, in the third embodiment, since the ultrasound diagnostic apparatus processing unit 1b is equipped with the envelope detecting unit and the sampling unit, the power consumption in the ultrasound transducer 2b becomes large as compared with the ultrasound diagnostic apparatus S in the second embodiment. However, the data transmission rate can be improved. For example, the total sum of data are obtained on the condition that the sampling frequency of sound ray data is 60 MHz, the number of transducers is 192, and the number of bits per one oscillator is 14, and in the case where 22 bits data at high order in the total sum of data are transmitted wirelessly, the data can be transmitted with a data transmission rate calculated as follows.

60×10⁶×22=1.32 (Gbps) (2)

As a result, as compared with the case where phasing addition is not conducted in an ultrasound probe, the data transmission rate can be reduced to 1/122.

As explained above, according to the first through third embodiments of the present invention, the ultrasound probe 2 (2a, 2b) is equipped with the error correcting code generating unit 208 which generates error correcting codes corresponding to ultrasound data, adds the error correcting coded generated by the error correcting code generating unit 208 to ultrasound data so as to produce transmission data. Then, the ultrasound probe 2 (2a, 2b) transmits the produced transmission data to the ultrasound diagnostic apparatus processing unit 1 (1a, 1b). Here, the ultrasound diagnostic apparatus processing unit 1 (1a, 1b) is equipped with the error correcting unit 103 which performs error correction processing to the ultrasound data contained in the transmission data based on the error correcting codes contained in the received transmission data, and the error ratio detecting unit 110 which detects the error ratio of data in the ultrasound data contained in the transmission data. Then, the ultrasound diagnostic apparatus processing unit 1 (1a, 1b) transmits the information corresponding to the error ratio detected by the error ratio detecting unit 110 to the ultrasound probe 2 (2a, 2b). Successively, the error correcting code generating unit 208 changes the ratio of error correcting codes to the ultrasound data in accordance with the information corresponding to the error ratio which the ultrasound probe 2 (2a, 2b) received, and generates error correcting codes. As a result, in the case where it is not likely to receive interference of noises easily and the error ratio in transmission data is low, the ratio of the magnitude of the data of error correcting codes added to ultrasound data to that of the ultrasound data is made small. Accordingly, the amount of data to be transmitted is decreased, so that the data transmission rate can be improved, and error correction can be conducted appropriately. On the other hand, in the case where the error ratio in transmission data becomes high, the ratio of the magnitude of the data of error correcting codes added to ultrasound data to that of the ultrasound data is made large, so that even under the environment where errors tend to be caused in ultrasound data, error correction can be conducted surely. Thereby, data can be transmitted with a suitable data transmission rate in response to the transmission state, and the transmission efficiency of data becomes good.

Further, according to the first embodiment of the present invention, the ultrasound probe 2 is equipped with the envelope detecting unit 206 which detects the envelope of the acquired reception signals. Then, the ultrasound probe 2 conducts sampling with a predetermined period for the envelope detected by the envelope detecting unit 206, and thereby generates ultrasound data. As a result, even if data are subjected to down sampling and transmitted wirelessly, the faithfulness of the waveform can be maintained. Accordingly, it can become possible to make the amount of data required for transmission small, and a data transmission rate can be raised. Further, in the ultrasound diagnostic apparatus processing unit, since ultrasound diagnostic images are produced based on the envelope data wirelessly transmitted from the ultrasound probe, image production processing is easy, and becomes excellent in general versatility in an ultrasound diagnostic apparatus processing unit.

Further, according to the first through third embodiments of the present invention, the ultrasound probe 2 (2a, 2b) is equipped with the wireless transceiver unit 210 which sets up the magnitude of the transmission output of transmission data in conformity with the information corresponding to the received error ratio, and transmits transmission data with the set-up magnitude of the transmission output. Accordingly, data transmission can be conducted with a proper magnitude of the transmission output in accordance with the transmission state, and then, when an error ratio is low, power consumption is suppressed, and when an error ratio is high, the wireless transmission stabilized more can be performed.

Further, according to the first through third embodiments of the present invention, when the error correcting unit 103 cannot conduct error correction for ultrasound data, the ultrasound diagnostic apparatus processing unit 1 (1a, 1b) conducts predetermined error processing for ultrasound data Successively, the ultrasound diagnostic apparatus processing unit 1 (1a, 1b) produces ultrasound diagnostic images based on the data obtained by the error processing. As a result, when errors for which error correction cannot be conducted occurs, suitable image production processing can be performed for such errors.

Further, according to the first through third embodiments of the present invention, when the error correcting unit 103 cannot conduct error correction for ultrasound data, the ultrasound diagnostic apparatus processing unit 1 (1a, 1b) is equipped with the display unit 107 which notifies the situation, it becomes possible to make users to recognize the situation that errors cannot be corrected.

The description in the embodiments of the present invention is an example of the ultrasound diagnostic apparatus according to the present invention, and the present invention is not limited to this example. Modification can be made suitably for the detail structures and detail operations of each function part which constitutes the ultrasound diagnostic apparatus.

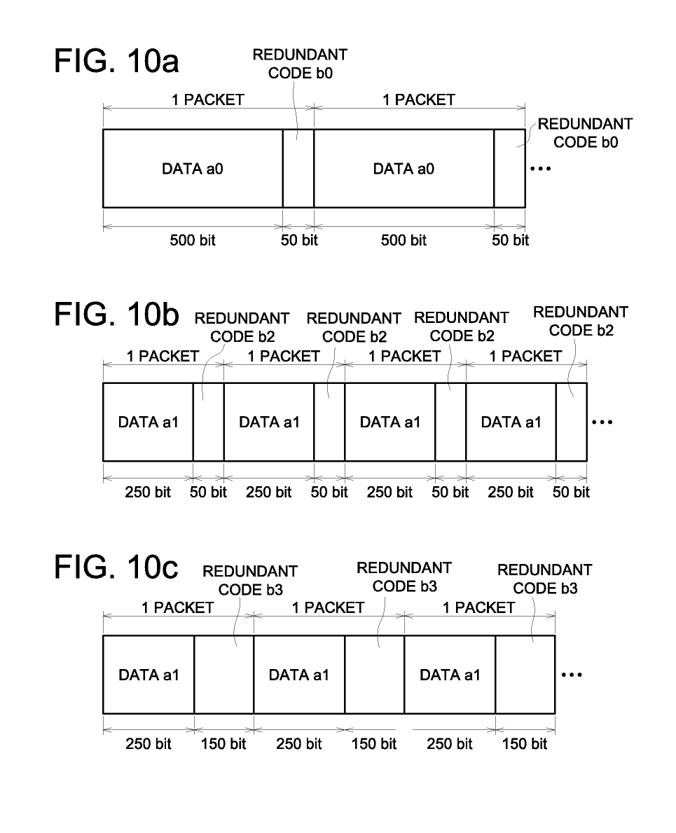

Further, in this embodiment, the ratio of the error correcting codes to ultrasound data is changed by changing of the code length of the error correcting codes added to the ultrasound data in accordance with an error rate. However, the ratio of the error correcting codes to ultrasound data may be changed such that, for example, as a result of detection of an error ratio after wireless transmission with the data structure shown in FIG. 10a, in the case where an error ratio is high, the data length of the ultrasound data a0 composed of 500 bits is decreased to 250 bits as shown in FIG. 10b, and then the redundant codes b2 with a data length of 50 bits is added to the ultrasound data a1 the data length of which has been decreased.

Further, the ratio of the error correcting codes to ultrasound data may be changed such that, as shown in FIG. 10c, the data length of the ultrasound data a0 composed of 500 bits is decreased to 250 bits with the name of ultrasound data a1, and then the redundant codes b3 with a data length of 150 bits is added to the ultrasound data a1.

Further, a packet length is fixed, and the data length of ultrasound data and the code length of redundant codes may be changed.

Furthermore, in this embodiment, the envelope data, sound ray data, or reception signal data are transmitted wirelessly. However, for example, ultrasound image data are produced in the ultrasound probe, and transmitted to the ultrasound diagnostic apparatus processing unit. Subsequently, ultrasound images are displayed based on the received ultrasound image data on the ultrasound diagnostic apparatus processing unit.

Further, in this embodiment, in the case where ultrasound data cannot be restored by error processing, predetermined error processing is made to be conducted. However, error processing is not conducted, and in the case where ultrasound data cannot be restored, it may be structured that images based on the ultrasound data are not displayed. Further, display of images may be suspended.

Furthermore, in this embodiment, in the case where ultrasound data cannot be restored by error processing, the display unit is made to perform as a notifying unit so as to display warning. However, for example, emitters, the notifying may be made such that light emitters such as LED (Light Emitting Diode), are made to emit light in a predetermined mode. Moreover, audio output units such as speakers are provided such that the notifying may be made by audio. Moreover, such notifying may not be conducted.

Further, in this embodiment, an error ratio is detected in an ultrasound diagnostic apparatus processing unit, error ratio information which shows the error ratio is transmitted to an ultrasound probe, and the code length of the error correcting codes is configured to be determined by the ultrasound probe. However, it may be structured that the code length of the error correcting codes corresponding to the error ratio is determined in an ultrasound diagnostic apparatus processing unit, and the resulting information may be transmitted to the ultrasound probe.

Further, in this embodiment, an error ratio is detected in the ultrasound diagnostic apparatus processing unit, and the error ratio information which shows the error ratio is transmitted to the ultrasound probe. However, for example, the trend of change of an error ratio is predicted by a predetermined calculation, and the results may be transmitted to an ultrasound probe. According to this, the error correction to ultrasound data can be more certainly performed.

Further, this embodiment discloses the example in which hard disks and nonvolatile memories of semi-conductors are used as media which can be read out by the computer with the programs according to the present invention. However, the present invention is not limited to this example. As another media which can be read out by the computer, potable type recording media, such as CD-ROM may be employed. Further, as media to provide the data of programs relating to the present invention via communication lines, carrier waves (carrier wave) may be also employed.

The above-mentioned preferable embodiment of the present invention will be summarized as follows.

Item (1) An ultrasound diagnostic apparatus comprises an ultrasound probe which outputs transmission ultrasound waves by driving signals towards an object to be examined, receives ultrasound waves reflected from the object so as to acquire reception signals, and generates ultrasound data based on the acquired reception signals; and a processing unit which produces an ultrasound diagnostic image based on the ultrasound data;
wherein the ultrasound probe and the processing unit transmit and receive data wirelessly,
wherein the ultrasound probe includes an error correcting code generating unit which generates error correcting codes corresponding to the ultrasound data, and the ultrasound probe adds the error correcting codes generated by the error correcting code generating unit to the ultrasound data so as to produce transmission data, and transmits the transmission data to the processing unit;
wherein the processing unit includes an error correcting unit which performs error correction processing for the ultrasound data contained in the received transmission data based on the error correcting codes contained in the transmission data, and an error ratio detecting unit which detects an error ratio of data in the ultrasound data contained in the transmission data, and the processing unit transmits information corresponding to the error ratio detected by the error ratio detecting unit to the ultrasound probe, and
wherein the error correcting code generating unit changes a ratio of error correcting codes to the ultrasound data in accordance with the information corresponding to the error ratio received by the ultrasound probe and generates error correcting codes.
   Item (2) The ultrasound diagnostic apparatus described in Item (1) is characterized in that the ultrasound probe includes an envelope detecting unit which detects an envelope of the acquired reception signals, and the ultrasound probe conducts sampling with a predetermined period and generates the ultrasound data from the envelope detected by the envelope detecting unit.
   Item (3) The ultrasound diagnostic apparatus described in Item (1) or (2) is characterized in that the ultrasound probe includes a wireless transmission unit which sets a magnitude of a transmission output of the transmission data in accordance with the received information corresponding to the error ratio and transmits the transmission data with the set magnitude of the transmission output.
   Item (4) The ultrasound diagnostic apparatus described in any one of Items (1) to (3) is characterized in that when the error correcting unit cannot perform error correction for the ultrasound data, the processing unit performs predetermined error processing for the ultrasound data, and produces an ultrasound diagnostic image based on data obtained by the error processing.
   Item (5) The ultrasound diagnostic apparatus described in any one of Items (1) to (4) is characterized in that when the error correcting unit cannot perform error correction for the ultrasound data, the processing unit includes an informing unit which informs the situation.
   According to the present invention, data can be transmitted with a proper data transmission rate in accordance with a transmission state.

What is claimed is:

1. An ultrasound diagnostic apparatus comprising:
an ultrasound probe which outputs transmission ultrasound waves based on drive signals toward an object to be examined, receives ultrasound waves reflected from the object so as to acquire reception signals, and generates ultrasound data based on the acquired reception signals; and
a processing unit which produces an ultrasound diagnostic image based on the ultrasound data;
wherein the ultrasound probe and the processing unit respectively includes a transceiver unit to transmit and receive data wirelessly,
wherein the ultrasound probe includes an error correcting code generating unit which generates error correcting codes corresponding to the ultrasound data, and the ultrasound probe adds the error correcting codes generated by the error correcting code generating unit to the ultrasound data so as to produce transmission data, and transmits the transmission data to the processing unit,
wherein the processing unit includes an error correcting unit which performs error correction processing for the ultrasound data contained in the received transmission data based on the error correcting codes contained in the transmission data, and an error ratio detecting unit which detects an error ratio of data in the ultrasound data contained in the transmission data, and the processing unit transmits information corresponding to the error ratio detected by the error ratio detecting unit to the ultrasound probe, and
wherein the error correcting code generating unit changes a ratio of error correcting codes to the ultrasound data in accordance with the information corresponding to the error ratio received by the ultrasound probe and generates error correcting codes.

2. The ultrasound diagnostic apparatus described in claim 1, wherein the ultrasound probe includes an envelope detecting unit which detects an envelope of the acquired reception signals, and a sampling unit which conducts sampling with a predetermined period and generates the ultrasound data from the envelope detected by the envelope detecting unit.

3. The ultrasound diagnostic apparatus described in claim 2, wherein the sampling unit changes a sampling period in accordance with the information corresponding to the error ratio received by the ultrasound probe and generates the ultrasound data.

4. The ultrasound diagnostic apparatus described in claim 1, wherein the ultrasound probe includes the transceiver unit which sets a magnitude of a transmission output of the transmission data in accordance with the information corresponding to the received error ratio and transmits the transmission data with the set magnitude of the transmission output.

5. The ultrasound diagnostic apparatus described in claim 1, wherein when the error correcting unit cannot perform error correction for the ultrasound data, the processing unit performs predetermined error processing for the ultrasound data, and produces an ultrasound diagnostic image based on data obtained by the error processing.

6. The ultrasound diagnostic apparatus described in claim 1, wherein the processing unit includes an informing unit which informs a situation that the error correcting unit cannot perform error correction for the ultrasound data.
